(19) United States
(12) Patent Application Publication
(10) Pub. No.: US 2022/0273863 A1
(43) Pub. Date: Sep. 1, 2022

(54) SUCTION HEAD FOR GENTLY SUCKING OFF THIXOTROPIC LIQUIDS

(71) Applicant: Georg-August-Universität Göttingen Stiftung Öffentlichen Rechts, Universitätsmedizin, Göttingen (DE)

(72) Inventor: Martin Friedrich, Bovenden (DE)

(21) Appl. No.: 17/747,343

(22) Filed: May 18, 2022

Related U.S. Application Data
(63) Continuation of application No. PCT/EP2019/081620, filed on Nov. 19, 2019.

(51) Int. Cl. A61M 1/00

(52) U.S. Cl. CPC A61M 1/87

(57) ABSTRACT

A suction head for sucking off a liquid containing organic components comprises an inner surface defining a main channel of the suction head. The main channel extends along a main axis of the suction head towards a suction connection of the suction head. Further, the suction head comprises suction holes entering into the suction head and feeding through the inner surface into the main channel, and flow-guiding devices. The flow guiding devices are configured for imparting a rotational component about the main axis on a flow of the liquid through the main channel, when the flow of the liquid which gets into the suction head through the suction holes is brought about by a negative pressure in the suction connection.

### CROSS REFERENCE TO RELATED APPLICATIONS

This application is a continuation of International Application PCT/EP2019/081620 with an international filing date of Nov. 18, 2019 entitled “Saugkopf zum schonenden Absaugen thixotroper Flüssigkeiten”.

### FIELD OF THE INVENTION

The invention relates to a suction head for sucking off a liquid containing organic components. Particularly, the invention relates to a suction head for sucking off a liquid containing organic components, the suction head comprising an inner surface defining a main channel of the suction head, the main channel extending along a main axis of the suction head towards a suction connection of the suction head, and suction holes entering into the suction head and feeding through the inner surface into the main channel.

The liquid containing organic components, which is to be sucked off, may particularly be a biological liquid, like for example blood, but also any other body fluid or any other fluid containing biological other organic components. The biologic components may, for example, be living cells, large organic molecules and complexes, like, for example, nucleic acid chains and proteins. The organic components may be dissolved or suspended in the liquid to be sucked off.

When sucking off blood out of an area of a surgical intervention in order to return it to the patient at which the respective surgical intervention is carried out, different problems occur. These problems include mixing the blood with ambient air in form of bubbles, damages to components of the blood, inclusive and particularly to red blood cells, white blood cells and thrombocytes due to shearing forces and a sucking suction head adhering to adjacent tissue of the patient. Ambient air mixed with the blood has to be removed, before the blood may be returned to the patient to avoid air embolism. If blood damaged by shearing forces is returned to a patient, this may result in incalculable damages, inter alia kidney failure, lung damages, thromboses, wound healing disorders, systemic inflammatory reactions. A sucking suction head adhering to tissue is connected with the danger of damages to this tissue. Further, with a intermittently adhering sucking suction head, sucked off blood may be subjected to very high shearing forces, whereas a permanently adhering sucking suction head is out of function.

Corresponding problems occur with sucking off other liquids containing complex organic components, like inter alia undesired activation, false activation, structure alterations to molecules like folding, denaturation, disintegration and the like.

### BACKGROUND OF THE INVENTION

International application publication WO 2012/092 948 A1 and United States patent application publication US 2013/0 324 954 A1 belonging to the same patent family disclose a suction device for suctioning liquids, particular for suctioning blood out of the area of a surgical intervention. The device comprises a suctioning element which is provided with at least one front suction opening at its distal end, several lateral suction openings and a pump connected to the suction device. The pump generates a suction vacuum in the suction element. A suction power of the pump effective at the suction openings is adjusted by means of a control device. The control device is connected to a sensor for acoustic waves which senses soundwaves and other vibrations generated by the suction device during its operation. When the control device, by means of the acoustic wave sensor, detects a characteristic soundwave pattern which corresponds to a slurping suction sound, it reduces the suction power at the suction openings, because the slurping suction sound indicates unfavorable suction conditions which are related to the danger of damages to blood components. Additionally, the control device is connected to a sensor for detecting a sucking adherence, and upon detecting a sucking adherence of the suction device with this sensor, the control device also reduces the suction power at the suction openings.

United States patent application publication US 2014/0 276 486 A1 discloses a cardiotomy suction tube system with multiple tips. Some of these tips comprise a hollow main body with a plurality of openings which allow for a fluid communication between the surroundings and an interior of the hollow main body. Further, these tips comprise a cylindrical extension which protrudes from hollow main body and to which a suction line is connected. The main body is capsule-shaped with rounded ends. One opening of the main body is provided at its distal end, the other openings are provided at its circumference.

U.S. Pat. No. 7,955,318 discloses a multipurpose large bore medical suction system relating to medical instruments comprising suction devices that are used to remove material from body cavities during medical procedures. The system is designed to adapt to large-bore medical vacuum sources such as the large-bore port of a collection canister. The system comprises a series of interchangeable tips. One of this tips has an atraumatic shape for reducing tissue trauma during the medical procedure. The atraumatic shape includes an essentially rounded surface, and this tip is also designated as a suction component with a blunt tip. The tip has a plurality of distal tip inlets and a plurality of lateral tip inlets which together form a fine suction field like a sieve.

International application publication WO 88/00 481 A1 discloses a surgical suction device having a perforated tip for removal of surgical debris with reduced clogging and with minimum trauma to adjacent tissue. Suction ports are arranged on the tip so that suction ports which remain unblocked when surgical debris lodges in other suction ports operate as a vacuum modulator facilitating the removal of the blockage. Further, the likelihood of blocking every suction port, thereby aspirating and damaging tissue, is greatly reduced. More particularly, suction ports are provided both at a front side end also at a backside of the conk-shaped tip which are orientated towards a center of the conk-shaped tip.

U.S. Pat. No. 5,827,218 discloses a suction tip for a surgical irrigation apparatus. The tip includes an outer tube having a distal end portion for communication with a surgical site and a proximal end for communication with a suction irrigation handpiece, and an inner tube extending in the outer tube and having an open distal end portion for communication with the surgical site and an open proximal end portion for communication with the handpiece. The suction head is configured to minimize the turbulence adjacent to sensitive organs during sucking off. A turbulence minimizing distal end region of the outer tube has a convexly rounded end with suction flow holes which extend through the wall of the outer tube into its hollow interior and which are arranged at distances in circumferential direction and axially. The suction flow holes are arranged in rows extending axially and arranged at distances in circumferential direction, wherein each row includes one hole pointing forward at the rounded end and six radially oriented holes.

U.S. Pat. No. 5,163,926 discloses a suction metering and mixing device for collecting body fluids such as blood and simultaneously mixing an anticoagulant therewith. The device includes a suction passage having an inlet end and an opposite end for connection to a vacuum supply. The anticoagulant flows through a supply tube to a position adjacent to the inlet of the suction passage where it is mixed with the blood entering the suction passage. A mixing cap in form of a half-sphere is positioned over the opening of the tube and the inlet of the suction passage. The mixing cap includes holes through which blood may enter into the device, and the cap forms a mixing chamber for bringing anticoagulant into fluid communication with blood as it is sucked into the suction passage.

United States patent application publication US 2017/0 224 887 A1 discloses a system for separating a flow of matter in which the flow of matter from a surgical instrument is tangentially supplied into a cylinder jacket-shaped or ring-shaped cavity. With respect to its cylinder axis, the cavity is oriented vertically. At an upper end of the ring-shaped cavity a suction port is provided for sucking off the flow, and at its lower end the matter separated from the flow is collected.

German patent application publication DE 196 50 407 A1 and U.S. Pat. No. 6,066,111 belonging to the same patent family disclose an apparatus for removing gas from blood, particularly in a blood flow which is sucked off a wound of a patient. A non-rotating centrifuge chamber has an inlet above and an outlet below with the chamber narrowing like a funnel between the inlet and the outlet. The inlet is oriented for directing the flow tangentially into the blood inlet and around the centrifuge chamber. The outlet is connected to a suction source for drawing off blood without reversal of the direction of rotation in the stream of blood in the chamber. The blood is further directed in the direction of flow of blood obliquely downward into the centrifuge chamber with an upwardly open angle to the axis rotation that is less than 90°.

It is known that blood is no so-called Newton liquid but a thixotropic liquid whose viscosity decreases with increasing shearing forces and flow velocities.

There still is a need of a suction head or tip which minimizes the problems described at the beginning in sucking off liquids containing organic components and particularly blood out of areas of surgical interventions.

### SUMMARY OF THE INVENTION

The present invention relates to a suction head for sucking off a liquid containing organic components. The suction head comprises an inner surface defining a main channel of the suction head. The main channel extends along a main axis of the suction head towards a suction connection of the suction head. Further, the suction head comprises suction holes entering into the suction head and feeding through the inner surface into the main channel, and flow-guiding devices. The flow-guiding devices are configured for imparting a rotational component about the main axis on a flow of the liquid through the main channel, when the flow of the liquid which gets into the suction head through the suction holes is brought about by a negative pressure in the suction connection.

Other features and advantages of the present invention will become apparent to one with skill in the art upon examination of the following drawings and the detailed description. It is intended that all such additional features and advantages be included herein within the scope of the present invention, as defined by the claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals designate corresponding parts throughout the several views.

FIG. 1 shows a suction head according in a longitudinal section along a main axis of the suction head.

FIG. 2 shows a detail of a further embodiment of the suction head in a schematic section orthogonal to its main axis.

FIGS. 3, 4 and 5 each show a detail of a further embodiment of the suction head in a schematic section orthogonal to its main axis; and

FIGS. 6 and 7 illustrate a special two-part formation of a suction connection in a further embodiment of the suction head in a longitudinal section along its main axis and in a cross-section orthogonal to its main axis.

### DETAILED DESCRIPTION

In a suction head for sucking off a liquid containing organic components, the suction head comprising an inner surface defining a main channel of the suction head, the main channel extending along a main axis of the suction head towards a suction connection of the suction head, and suction holes entering into the suction head and feeding into the main channel through the inner surface, flow guiding devices of the suction head are configured in such a way that the flow guiding devices impart a rotational component around the main axis on a flow through the main channel of a liquid entering into the main channel through the suction holes, the flow of the liquid being brought about by a negative pressure in the suction connection. In other words, the flow guiding devices result in that the flow of the liquid does not only run along the main axis of the suction head through the main channel, but also around this main axis. Assuming that the velocity of the liquid along the main axis of the suction head remains constant, the additional rotational component of the flow means a higher absolute value of the velocity of the flow. With thixotropic properties of the liquid containing organic components, this advantageously results in a reduced viscosity of the liquid. The viscous flow resistance of the liquid decreasing with decreasing viscosity does not only mean a lower flow resistance but also a quicker separation of air bubbles from the liquid. The rotational component of the flow of the liquid around the main axis and the related centrifugal forces may be so high that air sucked through the suction holes into the main channel together with the liquid concentrates in the area of the main axis and may be removed from there, whereas the liquid accumulates at the inner surface delimiting the main channel and may be sucked off separately from the air. Further, in the flow flowing on helical tracks trough the suction head, any discontinuities may be easily avoided that are connected with the danger of causing pressure steps or high shearing forces on the organic components of the liquid which may result in damages to these organic components.

Insofar as a negative pressure is mentioned here, this refers to a pressure below the pressure in the surroundings of the suction head.

Practically, the way, that is additionally covered by the flow of the liquid through the main channel due to the rotational component around the main axis, may be by at least 50% or at least 100% longer than the extension of the main channel along the main axis. The way of the flow through the main channel may even be extended to clearly more than twice the minimum way along the main axis, and practically, for example to at least three times, five times or even ten times this minimum way. Towards higher values, the extension of the way covered by the flow of the liquid through the main channel is naturally limited by the still present flow component along the main axis of the suction head. Thus, the way covered by the flow of the liquid due to the rotational component around the main axis will hardly be longer than 500 times the extension of the main channel along the main axis. Often it will not be more than 100 times longer.

The rotational component of the flow around the main axis may be caused or provided by different measures. For example, the flow guiding devices may comprise one or more of the following features.

The suction holes may each feed into the main channel in a feeding direction which, with respect to a circular arc around the main axis, comprises a tangential direction component. The feeding directions may exactly be tangential with respect to the circular arcs around the main axis, or they may have axial direction components along the main axis, particularly towards the suction connection, in addition to their tangential direction components.

The suction connection may branch off from the main channel in a branching-off direction which has a tangential direction component with respect to a circular arc around the main axis. This tangential direction component may once again be the only direction component of the branching-off direction, or it may be combined with an axial direction component.

An injection nozzle for an auxiliary liquid may inject into the main channel in an injection direction which has a tangential direction component with respect to a circular arc around the main axis. In case of blood as the sucked off liquid, the auxiliary liquid may, for example, be a heparin solution or any other liquid which is used to avoid a coagulation of the blood or to enhance its flow properties. It also applies for the injection direction of the injection nozzle for the auxiliary liquid that it may just have the tangential direction component or an additional axial direction component. An automatic dosage of the auxiliary liquid may be effected by a negative pressure present at the injection point of the injection nozzle into the main channel.

Helix-shaped or helical flow guiding elements may be arranged at the inner surface delimiting the main channel. These guiding elements may have the form of rips or fins protruding radially inwards from the inner surface and helically running around the main axis. The inner surface may further be provided with a lotus-effect which results in that an overflow resistance, i.e. the flow resistance of the flow flowing over the inner surface, is lower along a helical track around the main axis than parallel to the main axis. Besides the passive measures described up to here, the flow guiding devices may also include active measures, like for example an acceleration body which forms a part of the inner surface and which is rotated forth and back around the main axis. Such an acceleration body causes a rotational component of the flow around the main axis, if it is rotated with different rotation velocities in the one and the other rotation direction around the main axis, or if it has a, for example, scaly structured surface. A further active measure is an acceleration body arranged in the main channel and continuously rotated about the main axis. Such an acceleration body may even have a smooth surface.

The way additionally covered by the flow of the liquid through the main channel due to the rotational component around the main axis, when compared to the extension of the main channel along the main axis, may increase towards the suction connection. Such an increase may be achieved by a decreasing pitch of the helical tracks of the flow around the main axis or by an increasing diameter of the helical tracks. In other embodiments of the suction head, the way additionally covered by the flow of the liquid through the main channel due to the rotational component around the main axis, when compared to the extension of the main channel along the main axis, may decrease towards the suction connection. This may be achieved by at least one of an increasing pitch and a decreasing diameter of the helical tracks around the main axis.

Typically, a free flow cross-section of the suction head along the flow of the liquid towards the suction connection decreases resulting in an increase of the flow velocity of the flow. The decrease of the free flow cross-section of the suction head may be limited to the suction holes which may be trumpet-shaped to successively accelerate the liquid in the suction holes towards the main channel. The decrease of the free flow cross-section along the flow may be continued within the main channel. Here, the free flow cross-section may also be constant or even increase again to adjust the flow velocity of the sucked off liquid. In any case, it is preferred, if the free flow cross-section has a steady course, i. e. no step-like variations. Preferably, the variation of the free flow cross-section also has a steady course, such that the deviation of the free flow cross-section with respect to the way of the flow also has no steps.

Practically, each suction hole may have a free cross-sectional area decreasing from an outer surface up to the inner surface of the suction head. This free cross-sectional areas of the suction holes may decrease from the outer surface up to the inner surface by at least 50%, i. e. to a half in each suction hole. The decrease may also be by at least 67%, i. e. to about a third, or by at least 75%, i. e. to a quarter. In upward direction, the decrease of the free cross-sectional areas of the suction holes from the outer surface to the inner surface is naturally limited by the necessarily remaining free cross-sectional areas at the inner surface. Thus, the free cross-sectional areas of the suction holes from the outer surface up to the inner surface will hardly be decreased by more than 95%. Often, they will not be decreased by more than 90%.

With the suction holes, it is preferred, if one of the suction holes which enters into the suction head closer to the suction connection has a higher flow resistance for the liquid up to the main channel than another ones of the suction holes that enter into the suction head farther away from the suction connection. If the suction head is immersed into the liquid to be sucked off, it is often the case that only the suction holes at the distal end of the suction head that enter into the suction head farthest away from the suction connection are completely immersed into the liquid whereas suction holes which enter into the suction hole closer to the suction connection are free of liquid. In order to at least avoid a complete shorting of the suction holes at the distal end of the suction head by the air sucked into the free suction holes, the flow resistances of the suction holes, with respect to the positions of their entries into the suction head, preferably decrease towards the distal end of the suction head and increase towards the suction connection. Practically, that one of the suction holes which enters into the suction head closest to the suction connection has an by at least 50% increased flow resistance as compared to the suction holes which enters into the suction head farthest away from the suction connection. Preferably, the flow resistance is increased by at least 100%, i. e. at least doubled, or even by at least 200%, i. e. at least tripled. In upward direction, the increase of the flow resistance is delimited by the necessarily remaining function of the suction holes considered. Thus, an increase of the flow resistance by more than 900% is hardly suitable. Often, the flow resistance will not be increased by more than 400%.

In the suction head, the inner surface may at least partially be covered by a slip-coating reducing the overflow resistance for the liquid. Correspondingly, the suction holes may be lined with such a slip-coating. Suitable materials for the slip-coating are known to those skilled in the art. They provide a surface to which even a boundary layer of the liquid does not adhere, but slips due to high boundary surface tensions. In this way, the flow resistance which the liquid has to overcome when flowing through the suction holes and the main channel is significantly reduced.

The suction head may comprise a 3D-printed shaped body in order to provide the suction holes and also the main channel with its delimiting inner surface with a shape which may at least not easily be formed by a material removing or molding production method. The surface quality obtained by 3D-printing may not be sufficient for the suction head. This may be compensated for in that the surfaces of the 3D-printed shaped body are covered with a continuous smooth coating. Such a coating may, for example, be formed by dipping the shaped body into a corresponding coating material or by sucking in a corresponding coating material into the shaped body.

In one embodiment of the suction head, the suction connection comprises an inner partial connection connected to the main channel on the main axis, and an outer partial connection feeding through the inner surface into the main channel at a distance to the main axis. If the main channel is not completely filled with the liquid to be sucked off, ambient air sucked into the suction head together with the liquid accumulates in the area of the main axis, whereas the liquid guided on the helical tracks around the main axis flows over the inner surface delimiting the main channel. Thus, the air may purposefully be removed via the inner partial connection, and the liquid may purposefully be sucked off via the outer partial connection. A switch-over device that switches over between the two partial connections may be provided and configured such that it opens the inner partial connection connected to the main channel on the main axis and closes the outer partial connection which feeds through the inner surface into the main channel at the distance to the main axis, when ambient air is sucked off into the suction head.

At its distal end facing the suction connection, the main channel of the suction head will typically be closed by at least a third or a half in order to avoid a primary axial flow through the main channel. For this purpose, the main channel may even be closed by two thirds or three quarters or completely at its distal end facing away from the suction connection. As long as the main channel at its distal end facing away from the suction connection is not completely closed, single suction holes may feed through the inner surface into the main channel at the end of the distal end of the main channel. Preferably, these suction holes feed the sucked in liquid on a helical track into the main channel and are of a helical design for this purpose. Even if an axial suction hole feeds into the main channel on the main axis, the flow guiding devices impart the rotational component on the flow of the sucked off liquid, preferably already when the liquid passes through this axial suction hole.

It is to be understood that the suction head may be used as a part of a suction device as it is, for example, known from WO 2012/092 948 A1 and in which the suction power is controlled depending on the signal of a soundwave sensor. Thus, the suction head may have such a soundwave sensor.

Referring now in greater detail to the drawings, the suction head 2 or suction tip depicted in FIG. 1 in a section along its main axis 1 comprises an inner surface 3 which delimits a main channel 4 extending along the main axis 1. At a distal end 5 of the suction head 2, the main channel 4 is closed. Several suction holes 6 feed into the main channel 4. At a proximal end 8 of the suction head 2, a suction connection 7 is connected to the main channel 4. The suction connection 7 serves for connecting the suction head 2 via a separation device to a vacuum source which is not depicted here. The separation device serves for separating a liquid containing organic components, which is sucked off by means of the suction head 2.

The suction holes 6 enter into the suction head 2 through an outer surface 9 which defines the outer dimensions of the suction head 2. Free cross-sectional areas of the suction holes 6 decrease from the outer surface 6 up to the inner surface 3. Further, the free cross-sectional areas of the suction holes 6 decrease with their distance to the distal end 5. This means that the suction holes 6 that are closest to the suction connection 7 have the smallest free cross-sectional areas. Further, the suction holes 6 that are closest to the suction connection 7 are longer than the suction holes 6 that are closer to the distal end 5, because a shaped body 10 of the suction head 2 through which the suction holes 6 extend towards the main channel 4 has outer dimensions of a truncated cone. In this way it is avoided that then, when only the suction holes 6 close to the distal end 5 are dipped into a liquid to be sucked off, these suction holes 6 are shorted by the other suction holes 6 sucking in ambient air.

The shaped body 10 may, for example, be produced by 3D-printing. The surfaces of the shaped body 10 are provided with a continuous smooth coating 11 which forms the inner surface 3 and the outer surface 9 and lines the suction holes 6. The coating 11 may particularly be made as a slip-coating which strongly reduces an overflow resistance of the liquid to be sucked off.

The suction head 2 according to FIG. 1 includes flow guiding devices which impart a rotational component around the main axis 1 on a flow of the sucked off liquid entering into the main channel 4 through the suction holes 6, the flow being brought about by a negative pressure in the suction connection 7. This means that the flow through the main channel 4 passes through the main channel 4 on helical tracks around the main axis 1. Here, the rotational component of this flow is so big that the sucked off liquid in the main channel 4 contacts and covers the inner surface 3 even if ambient air is sucked through the suction holes 6 into the main channel 4 together with the liquid. Thus, mixing the sucked off liquid with this air is avoided.

It belongs to the flow guiding devices of the suction head 2 in the embodiment according to FIG. 1 that the suction holes 6 are not oriented radial to the main axis 1, but at an offset thereto, that helical flow guiding elements 12 are arranged at the inner surface 3 and that the suction connection 7 laterally branches-off at an offset.

Besides the shaped body 10, the suction head 2 according to FIG. 1 includes a tube piece 13 connected to the shaped body 10. The tube piece 13 may be longer or shorter than depicted and one part with the shaped body 10. A multipart construction of the suction head 2 may ease its cleaning and sterilization after use.

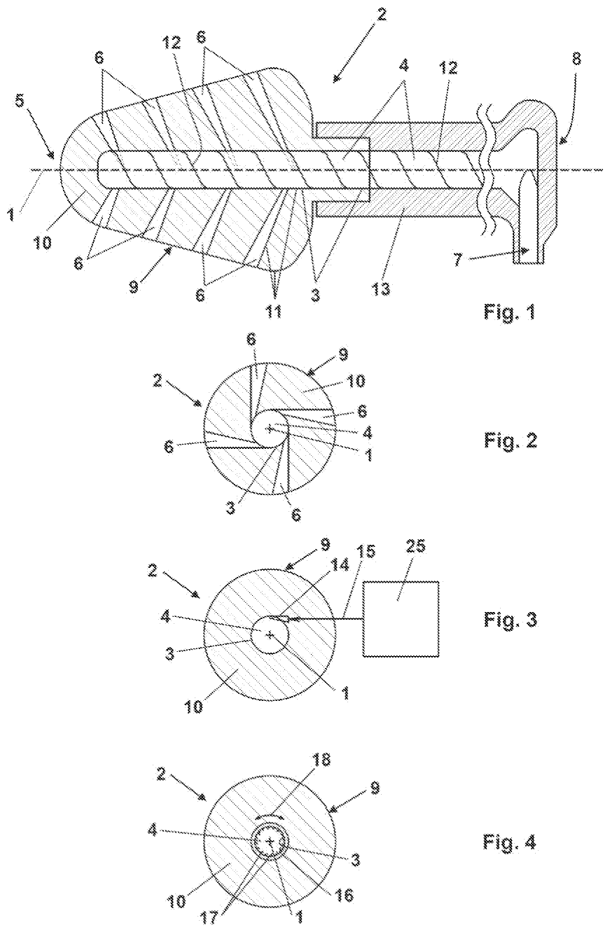

The section orthogonal to the main axis 1 through another embodiment of the suction head 2 depicted in FIG. 2 shows four suction holes 6 which each have a free cross-sectional area decreasing from the outer surface 6 up to the inner surface 3, and which each feed through the inner surface 3 into main channel 4 tangentially with respect to a circular arc around the main axis 1. In this way, a twist around the main axis 1 is imparted on the liquid sucked into the main channel 4 already by the suction holes 6 such that the liquid moves along the main axis 1 through the main channel 4 on the already mentioned helical tracks around the main axis 1.

The schematic cross-section through a further embodiment of the suction head 2 according to FIG. 3 shows an injection nozzle 14 for an auxiliary liquid 15, like for example a liquid anticoagulant, if the sucked off liquid is blood. The injection nozzle 14 feeds into the main channel 4 tangentially with respect to the circular arc around the main axis 1 defined by the inner surface 3. Auxiliary liquid 15 injected into the main channel 4 through the injection nozzle 14 by means of a pump 25 is deflected by the inner surface 3 so that it moves through the main channel 4 on the helical tracks around the main axis 1 and takes the liquid which has been sucked off into the main channel 4 along the helical tracks.

A further active measure of the flow guiding devices in the suction head 2 is illustrated in FIG. 4. Here, a ring-shaped acceleration body 16 is arranged within the shaped body 10. The acceleration body 16 at least partially forms the inner surface 3 which delimits the main channel 4. In the area of the acceleration body 16, the inner surface 3 is provided with scales 17 inclined against the circumferential direction around the main axis 1. Practically, these scales may be designed such as to achieve a so-called lotus-effect for the liquid to be sucked off. If the acceleration body 6 is then rotated forth and back around the main axis 1 as indicated by a double arrow 18, wherein its movement in the direction of the desired rotational component of the sucked off liquid may be slower than its movement against this rotational component, the sucked off liquid is accelerated by the acceleration body 16 in the direction of the desired rotational component.

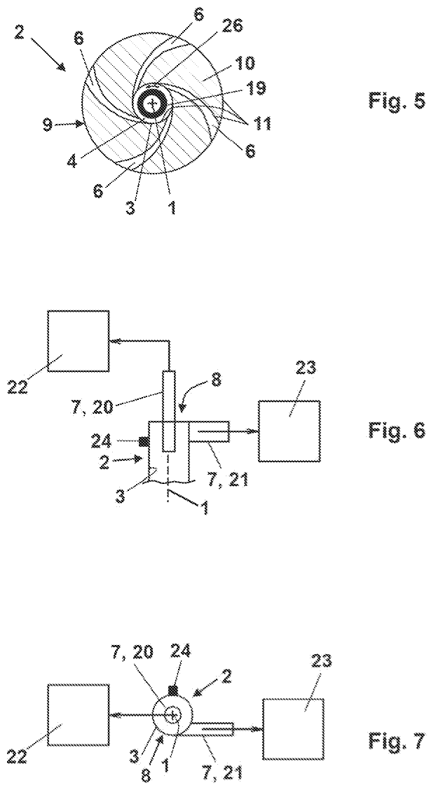

The embodiment of the suction head 2 illustrated in FIG. 5 comprises another acceleration body 19 rotationally driven about the main axis 1. This acceleration body 19 continuously rotates in the direction of a rotation arrow 26 within the interior of the main channel 4. The remaining free cross-section of the main channel 4 between the inner surface 3 and the acceleration body 19 is ring-shaped. In other words, the main channel 4 around the acceleration body 19 is a ring channel. In this ring channel, the rotational component in direction of the rotation arrow 26 is imparted on the flow of the sucked off liquid by means of the rotating acceleration body 19. If this acceleration body 19 is provided with a screw-shaped surface contour, it may also be used to convey the sucked off liquid in the direction of the main axis 1, i. e. to support the flow towards the suction connection or to even originally cause this flow like a suction turbine. Further, FIG. 5 shows a helical course of the suction holes 6 which also have cross-sectional areas decreasing from the outer surface 9 up to the inner surface 3. Such a helical course may hardly be provided by a molding method or a material removing method of producing the suction head 2. However, the shaped body 10 through which the suction holes 6 pass may be produced by 3D-printing and then be provided with the smooth coating 11 for enhancing its surface quality.

FIGS. 6 and 7 illustrate how to separately remove the sucked off liquid, on the one hand, and ambient air sucked in with the sucked off liquid, on the other hand, at the distal end 8 of the suction head 2 by means of two separate partial connections 20 and 21 of the suction connection 7 which lead to two separate vacuum sources 22 and 23. Via the partial connection 20 of the suction connection 7, the vacuum source 22 sucks off the air accumulated in the area of the main axis 1, whereas, via the partial connection 21 of the suction connection 7, the vacuum source 23 sucks off the liquid accumulated at the inner surface 3. A soundwave sensor 24 at the suction head 2 may detect air borne or structure born soundwaves. The vacuum sources 22 and 23 can be controlled depending on the signal of the soundwave sensor 24. Practically, if detected soundwaves indicate that ambient air is sucked into the suction head 2, the vacuum source 22 may suck off via the partial connection 20 primarily or even exclusively. If, on the other hand, the signal of the soundwave sensor 24 indicates that only liquid to be sucked off is sucked into the suction head 2, the vacuum source 23 may suck off via the partial connection 21 primarily or even exclusively. In this way, not only pure air, but also air with foamed sucked off liquid which, due to its lower density and the rotational component of its flow through the main channel 4, also accumulates in the area of the main axis 1 and thus separates from the liquid at the inner surface 3 may be sucked off via the partial connection 20. Further, if the sucked off liquid is blood, the ‘best’ blood, i. e. sane cells are primarily found in the outer layer at the inner surface 3, because sane erythrocytes have a higher mass than damaged blood components and thus, due to the centrifugal forces caused by the rotational component of the flow, accumulate at the inner surface 3.

Many variations and modifications may be made to the preferred embodiments of the invention without departing substantially from the spirit and principles of the invention. All such modifications and variations are intended to be included herein within the scope of the present invention, as defined by the following claims.

We claim:

1. A suction head for sucking off a liquid containing organic components, the suction head comprising
an inner surface defining a main channel of the suction head, the main channel extending along a main axis of the suction head towards a suction connection of the suction head,
suction holes entering into the suction head and feeding through the inner surface into the main channel, and
flow-guiding devices configured for imparting a rotational component about the main axis on a flow of the liquid through the main channel, when the flow of the liquid which gets into the suction head through the suction holes is brought about by a negative pressure in the suction connection.

2. The suction head of claim 1, wherein a way covered by the flow of the liquid through the main channel, due to the rotational component about the main axis, is by at least one of 50%, 100%, 200%, 400% and 900% longer than the extension of the main channel along the main axis.

3. The suction head of claim 1, wherein the flow guiding devices comprise at least one of the following features:
at least some of the suction holes each feed into the main channel in a feeding direction having a tangential direction component with respect to a circular arc around the main axis,
at least a partial connection of the suction connection branches off from the main channel in a branching-off direction having a tangential direction component with respect to a circular arc around the main axis,
an injection nozzle for an auxiliary liquid injects into the main channel in an injection direction having a tangential direction component with respect to a circular arc around the main axis,
helical flow guiding elements are arranged on the inner surface,
an acceleration body forming a part of the inner surface is configured for being rotated forth and back around the main axis,
an acceleration body arranged in the main channel is configured for being continuously rotated about the main axis.

4. The suction head of claim 1, wherein additionally ways which are covered by the flow of the liquid through the main channel due to the rotational component about the main axis per unit of extension of the main channel along the main axis increase towards the suction connection.

5. The suction head of claim 1, wherein additionally ways which are covered by the flow of the liquid through the main channel due to the rotational component about the main axis per unit of extension of the main channel along the main axis decrease towards the suction connection.

6. The suction head of claim 1, wherein a free flow cross-section of the suction head comprises a steady course along the flow of the liquid towards the suction connection.

7. The suction head of claim 1, wherein each of the suction holes comprises a free cross-sectional area decreasing towards the inner surface.

8. The suction head of claim 7, wherein the free cross-sectional area of each of the suction holes decreases from an outer surface of the suction head up to the inner surface by at least one of 50%, 67% and 75%.

9. The suction head of claim 1, wherein one of the suction holes which enters into the suction head closer to the suction connection than another ones of the suction holes has a higher flow resistance for the liquid up to the main channel than the other ones of the suction holes.

10. The suction head of claim 9, wherein the higher flow resistance of that one of the suction holes which enters into the suction head closest to the suction connection is by at least one of 50%, 100% and 200% higher than a flow resistance of that other one of the suction holes which enters into the suction head farthest away from the suction connection.

11. The suction head of claim 1, wherein the inner surface is at least partially covered with a slip-coating reducing its overflow resistance for the liquid.

12. The suction head of claim 1, wherein the suction holes are lined with a slip-coating reducing its overflow resistance for the liquid.

13. The suction head of claim 1, wherein the suction head comprises a 3D-printed shaped body whose surfaces are provided with a continuous smooth coating.

14. The suction head of claim 1, wherein the suction connection comprises an inner partial connection connecting to the main channel on the main axis, and an outer partial connection feeding through the inner surface into the main channel at a distance to the main axis.

15. The suction head of claim 14, wherein a switchover device which switches over between the two partial connections is configured for opening the inner partial connection and closing the outer partial connection when air is sucked off through the suction head.

16. The suction head of claim 1, wherein the main channel, at its end facing the suction connection, is closed by the inner surface over at least one of 33%, 50%, 67% and 75% of its free cross-sectional area in front of this end.

17. The suction head of claim 1, wherein the main channel, at its end facing the suction connection, is completely closed by the inner surface.
